(19) United States
(12) Patent Application Publication
(10) Pub. No.: US 2011/0070657 A1
(43) Pub. Date: Mar. 24, 2011

(54) DETECTING IONS AND MEASURING ION CONCENTRATIONS

(75) Inventors: Lee Josephson, Reading, MA (US); Sonia Taktak, Cambridge, MA (US)

(73) Assignee: THE GENERAL HOSPITAL CORPORATION, Boston, MA (US)

(21) Appl. No.: 12/673,650

(22) PCT Filed: Aug. 18, 2008
(86) PCT No.: PCT/US08/73515

§ 371 Date: Dec. 8, 2010

Related U.S. Application Data
(60) Provisional application No. 60/956,656, filed on Aug. 17, 2007.

(51) Int. Cl. G01N 33/53; C07C 233/01; C07F 1/08; C07D 323/00; C07F 3/06

(52) U.S. Cl. 436/501; 564/160; 556/110; 549/352; 549/353; 546/12

(57) ABSTRACT

The present inventions include methods and compositions for detecting the presence of ions and measuring the level or concentration of ions in a sample by nuclear magnetic resonance (NMR) using magnetic particles. In particular, the inventions include the preparation and use of magnetic particles having synthetic ion chelators covalently bound to their surfaces. In the presence of target ions, the surface-modified magnetic particles form clusters, which can be monitored by NMR relaxation measurements. The relaxation times can then be used to detect specific ions and determine their concentration. The described methods, compositions, and devices are useful for a variety of applications including biomedical applications in diagnostics and imaging.

### TECHNICAL FIELD

This disclosure relates to methods and compositions for detecting the presence of ions, and more particularly to measuring ion concentrations in samples.

### BACKGROUND

Magnetic particles, such as nanoparticles and microparticles, have emerged as valuable tools in numerous biotechnological applications. For example, certain magnetic particles have been used as magnetic resonance imaging (MRI) contrast agents and for nuclear magnetic resonance (NMR)-based sensing applications, in part due to their high relaxivities (change in water proton relaxation rate per mM of iron) and the ability to remain in suspension indefinitely. In some embodiments, certain magnetic particles can be used for applications requiring magnetic manipulation or extraction of the particles, such as immunoassays or cell sorting, because they can be more readily manipulated by the inhomogeneous magnetic fields of hand held magnets.

Magnetic nanoparticle-based assays can be used to detect oligonucleotides, proteins, viruses, and small molecules, with very high sensitivity and little or no sample preparation. Magnetic nanoparticle-based assays have also been employed as a component of sensors. In the presence of an intended binding target (or analyte), such nanoparticles can self-assemble, resulting in a change of relaxation time of surrounding water protons, which can be detected by NMR as described, for example, in Perez, Chembiochem., 2004, 5(3):261; and Perez, Nature Biotechnology, 2002, 20(8):816.

Two types of ion sensors using NMR detection of ions have been reported. The first type of ion sensor includes one or more low molecular weight gadolinium or europium chelates as contrast agents. Typically, such sensors include a contrast agent platform like DTPA (diethylenetriaminepentaacetic acid), although other platforms have been used as well, and one or multiple pendant chelators specific to the ion of interest. Binding of the target ion to the pendant chelator(s) modulates water access to the paramagnetic center resulting in a change in longitudinal relaxation time (T1). Such ion-sensitive MRI sensors have been described for the detection of Ca²⁺, Zn²⁺, and Cu²⁺, for example, in Li et al., J. Amer. Chem. Soc., 1999, 121:1413-1414; Li et al., Inorganic Chem., 2002, 41:4018-4024; Hanaoka et al., J. Chem. Soc. Perkin Transactions, 2001, 2:1840-1843; Hanaoka et al., Chem. Biol., 2002, 9:1027-1032; Trokowski et al., Angewandte Chemie-Int'l Ed., 2005, 44:6920-6923; and Que, et al., J. Amer. Chem. Soc., 2006, 128:15942-15943. However, these systems yield smaller changes in relaxivity than particle-based systems, and can thus be less sensitive.

A second type of ion sensor includes streptavidin-coated iron oxide nanoparticles to detect calcium ions, as described, for example, in Atanasijevic et al., Proc. Nat. Acad. Sci. U.S.A., 2006, 103:14707-14712. In this case, two populations of nanoparticles were generated by attaching the biotinylated calcium-binding protein calmodulin to a first population and the biotinylated target peptide M13 to a second population, via biotin/streptavidin binding. A mixture of the two populations of nanoparticles bearing calmodulin or the M13 domain was used to monitor calcium levels. In the presence of calcium, calmodulin binds to M13 resulting in clustering of the nanoparticles causing a change of the transverse relaxation time (T2). The strategy used in this case is based on protein/protein interaction, not very different from antibody/antigen interactions used in other nanoparticle-based NMR sensors and cannot easily be generalized to the detection of other ions.

### SUMMARY

This disclosure relates generally to NMR-based methods and compositions for detecting specific ions in liquid samples and measuring the concentration of the ions. The invention is based, in part, on the discovery that ion-binding molecules can be covalently bound to magnetic particles, which when exposed to an ionic analyte in a solvent, can aggregate as the ion-binding molecules bind to the analyte and affect the relaxation properties of the surrounding solvent. A change in the relaxation properties can indicate the presence of the ionic analyte and can be correlated with the concentration of the ionic analyte. The ion-binding particle does not contain biological molecules or derivatives thereof, rather, the particle includes covalently bound synthetic molecules.

In one aspect, the disclosure features ion-binding particles including a magnetic particle M; and at least one ion-chelating molecule Y covalently linked to the magnetic particle.

In another aspect, the disclosure features methods of detecting specific ions in samples by obtaining a first sample including a specific ion; contacting a sample with a plurality of ion-binding particles as described herein for a time and under conditions sufficient to allow the formation of ion/ion-binding particle complexes; measuring a relaxation time of the sample; and comparing the relaxation time of the sample with a relaxation time of a reference. A difference between the relaxation time of the sample and the relaxation time of the reference indicates the presence of the specific ion in the sample.

In yet another aspect, the disclosure features devices including a plurality of ion-binding particles enclosed within a semipermeable wall that allows the passage of an ion or ions that can be chelated by the ion-chelating molecule Y, but does not allow the passage of the ion-binding particles.

Embodiments can include one or more of the following features.

The ion-binding particle can include a moiety of Formula I linked to the magnetic particle M via one or more covalent bonds:

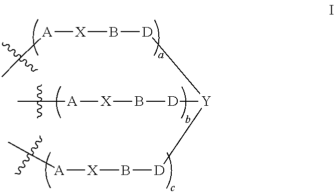

wherein

A is NHCO, CONH, S, O, or NR^{a};

X is absent or C₁₋₁₀ alkyl, C₁₋₆ haloalkyl, C₂₋₆ alkenyl, C₂₋₆ alkynyl, aryl, heteroaryl, cycloalkyl, or heterocycloalkyl, wherein said C₁₋₁₀ alkyl, C₁₋₆ haloalkyl, C₂₋₆ alkenyl, C₂₋₆ alkynyl, aryl, heteroaryl, cycloalkyl, or heterocycloalkyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

B is absent or a spacer;

D is absent, NHCO, CONH, S, O, or NR^{a};

Y is an ion-chelating molecule;

R^{a} is H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

a, b, and c are each independently 0 or 1; and

a+b+c is greater than or equal to 1.

In some embodiments, A is NHCO or CONH. In some embodiments, D is absent, NHCO, or CONH. In some embodiments, X is absent or C₁₋₁₀ alkyl. In some embodiments, X is absent or CH₂. In some embodiments, X is absent. In some embodiments, the spacer is alkyl interrupted by one or more O, NR^{a}, S, SO, SO₂, C(O)O, OC(O), NHCO, CONH, SC(O), or C(O)S, said alkyl is optionally terminated with one or two O, NR^{a}, S, SO, SO₂, C(O)O, OC(O), NHCO, CONH, SC(O), or C(O)S. In some embodiments, R^{a} is H or C₁₋₁₀ alkyl.

In some embodiments, Y is:

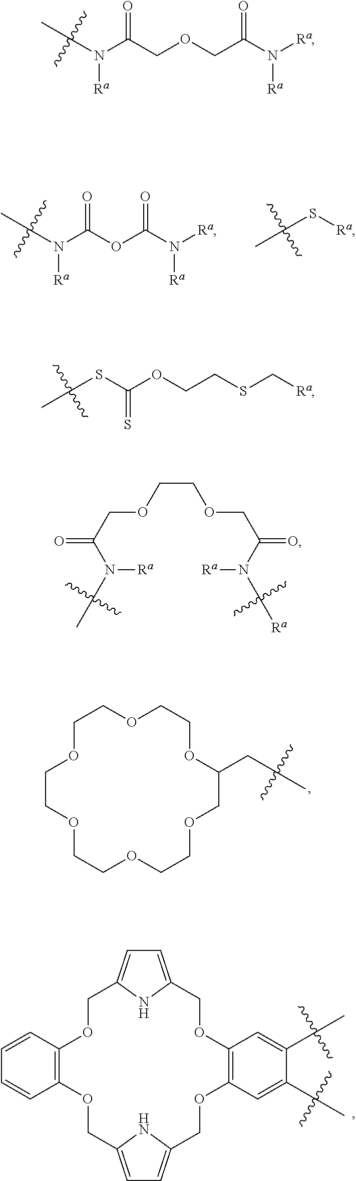

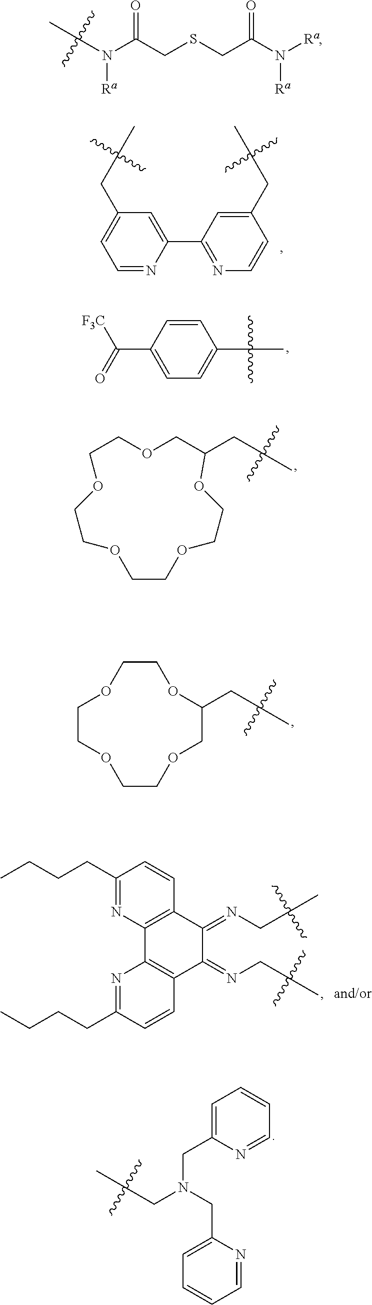

In various embodiments, Y can be a calcium-chelating molecule, a magnesium-chelating molecule, a copper-chelating molecule, a potassium-chelating molecule, a sodium-chelating molecule, a cesium-chelating molecule, and/or a zinc-chelating molecule.

The magnetic particles can have a maximum dimension of less than or equal to about one micron, for example, from about 15 to about 750 nm, e.g., 25 to 500 nm, or 50 to 250 nm. The magnetic particles can be or include a superparamagnetic material. In some embodiments, the magnetic particles can be or include one or more magnetic metal oxides. The particles can include from 1 to about 200 moieties of Formula I (e.g., from 10 to about 100 moieties of Formula I, from 25 to about 75 moieties of Formula I).

In some embodiments, the reference is a control sample free of the specific ion. In some embodiments, the reference is contacted with a plurality of magnetic particles M that are the same as those used in the ion-binding particles, but without the ion-chelating molecule Y. In some embodiments, the relaxation times of the sample and the reference are converted into data, and the data of the relaxation time of the sample is compared to the data of the relaxation time of the reference. The data of the relaxation time of the reference can be a calibration curve.

The methods can include measuring a concentration of the detected ion. The difference between the relaxation time of the sample and the relaxation time of the reference can correlate with a concentration of the ion in the sample.

The samples can include bodily fluids (e.g., blood, serum, and/or urine), or they can include water, e.g., drinking water, wastewater, chemical solutions, and paper slurry.

In some embodiments, the samples include an ion-binding particle concentration of at least 0.1 mM (e.g., at least 0.4 mM). A ratio of the relaxation time of the reference to the relaxation time of the sample can decrease upon formation of ion/ion-binding particle complexes.

The ion/ion-binding particle complexes can include two or more ion-binding particles. In some embodiments, formation of ion/ion-binding particle complexes is reversible upon addition of a competing chelating agent. The competing chelating agents can be EDTA, EGTA, DTPA, NTA acid, o-phenanthroline, dimercaptopropanol, and/or salicylic acid. In other embodiments, formation of ion/ion-binding particle complexes is non-reversible.

In some embodiments, the methods further include obtaining a device including a semipermeable wall that allows passage of the specific ion, but not the passage of the ion-binding particles; enclosing the ion-binding particles within the device; and allowing formation of the ion/ion-binding particle complexes within the device. The methods can include implanting the device in a subject, such as an animal or human subject. Alternatively, the devices can be immersed in the sample, e.g., when the sample is water, wastewater, a chemical solution, or the like.

As used herein, the term “magnetic” refers to materials of high positive magnetic susceptibility such as paramagnetic or superparamagnetic compounds and magnetite, gamma ferric oxide, or metallic iron.

As used herein, the term “paramagnetic” refers to particles that have a relatively high and positive magnetic susceptibility, but exhibit no magnetic moment in the absence of a magnetic field. Paramagnetic particles do not exhibit magnetic saturation.

As used herein, the term “superparamagnetic” refers to magnetic materials that exhibit magnetic properties in a magnetic field with no residual magnetism once removed from the magnetic field, that exhibit higher magnetic susceptibility than paramagnetic materials, and that show magnetic saturation (e.g., reaches a plateau magnetic value as the magnetic field is increased).

As used herein, the term “solvent” includes water, buffers, and organic solvents.

As used herein, the term “selective binding” refers to a preferential binding to a particular ionic analyte in the presence of other substances.

The invention provides a number of advantages including the following.

The magnetic particles can have superior properties compared with existing ion selective electrodes. The magnetic particles can have decreased time-dependent fouling of the surface, as the ion-binding particles are diluted into an assay fluid and there is no solid phase, electrode, or membrane. For example, when ion-binding particles are employed as components of a semipermeable device, the semipermeable device can have an increased lifetime compared to sensors with electrodes due to decreased surface fouling.

The magnetic particles can extend current magnetic particle/NMR technology to a broad new class of analytes such as ions. The assay methods are versatile, as many chemically different types of analytes, such as ions, can be,detected.

In some embodiments, the magnetic particles can be used in implantable sensors. For example, the magnetic particles can be used in methods of detecting ions and determining their concentrations in living organisms by using remote, implantable sensors, e.g., as described in U.S. application Ser. No. 11/431,247.

In some embodiments, the ion-selective magnetic particles can be custom-designed by adapting chelators used in the design of ion selective electrodes. The methods can be relatively cheap and amenable to a wide range of ions, for example, when compared with ion-binding proteins and peptides, which can be more expensive and available for use with fewer ions.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a schematic diagram of a device used to enclose the new ion-binding particles with a semi-permeable wall.

FIG. 2 is a graph showing the change in T2 relaxation time in presence of calcium ion at different nanoparticle concentrations.

FIG. 3 is a graph showing the change in relaxation time in presence of calcium. [Fe]=0.4 mM.

FIG. 4 is a graph showing the change in relaxation time in presence of calcium. [Fe]=0.2 mM. ▴ with complexing agent EDTA; ▪ no EDTA.

FIG. 5 is a graph showing the relative changes in relaxation times in presence of common interfering ions.

Like reference symbols in the various drawings indicate like elements.

### DETAILED DESCRIPTION

The present disclosure provides NMR-based methods and compositions for detecting the presence of and measuring the concentration of ions using magnetic particles, opening up a new class of analytes that can be detected using the same instruments and assay formats as those used in other NMR-based methods using magnetic particles. In particular, this disclosure describes the preparation and use of magnetic particles functionalized with (e.g., covalently bound to) ion-chelating molecules on the surface of the particles. When exposed to a test sample, for example, a solution including an ionic species (e.g., lithium, sodium, potassium, rubidium, cesium, francium, beryllium, magnesium, calcium, strontium, barium, radium, silver, samarium, lead, cesium, ammonium, copper, cadmium, carbonate, phosphate, and/or zinc ions), two or more functionalized magnetic particles can bind to the same target ion to form particle aggregates, which can cause a detectable change in the relaxation properties, such as the T2 properties, of the solvent (e.g., H₂O). Without wishing to be bound by theory, it is believed that there are at least two possible general mechanisms by which ion binding to the magnetic particles of the invention can affect particle aggregation and subsequently water relaxation properties. Ion binding can affect the charge or surface potential of particles, which is the repulsive force between particles. As the repulsive force decreases, for example, particles can aggregate. In some embodiments, an ion can bind directly to chelating groups on one or more magnetic particles, forming a bridge between the two particles and thereby aggregate the particles.

### Ion-Binding Particles

The new methods use new ion-binding particles that include a magnetic particle, e.g., a superparamagnetic or paramagnetic particle, and one or more ion-chelating molecules linked to its surface in such a way that two or more of the particles can bind to the same ion.

A key feature of the new methods is that they provide a general process through which magnetic particles can be designed with ion-binding surface moieties. Ion-selective magnetic particles are obtained by linking ion-chelating molecules, e.g., those previously known to work on ion electrodes, to the surface of particles. This provides access to an immense and successful range of literature on the design of ion-selective electrodes. This literature provides ion-chelating molecule candidates for designing magnetic particles with diverse types of ion binding surfaces, as described in further detail in the Examples below.

In general, the ion-binding magnetic particles include a magnetic particle M, and one or more ion-chelating molecules Y linked to the magnetic particle via one or more covalent bonds (represented by the solid and dashed lines).

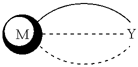

In some embodiments, the ion-binding particles can include a moiety of Formula I covalently bound to the magnetic particle M:

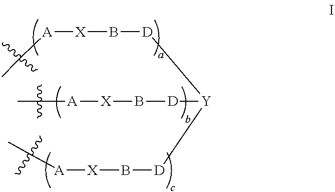

wherein

A is NHCO, CONH, S, O, or NR^{a};

X is absent, C₁₋₁₀ alkyl, C₁₋₆ haloalkyl, C₂₋₆ alkenyl, C₂₋₆ alkynyl, aryl, heteroaryl, cycloalkyl, or heterocycloalkyl, wherein said C₁₋₁₀ alkyl, C₁₋₆ haloalkyl, C₂₋₆ alkenyl, C₂₋₆ alkynyl, aryl, heteroaryl, cycloalkyl, or heterocycloalkyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

B is absent or a spacer;

D is absent, NHCO, CONH, S, O, or NR^{a};

Y is an ion-chelating molecule;

R^{a} is H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;

a, b, and c are each independently 0 or 1; and

a+b+c is greater than or equal to 1.

In some embodiments, A is NHCO or CONH.

In some embodiments, D is absent, NHCO, or CONH.

In some embodiments, D is absent.

In some embodiments, X is absent, C₁₋₁₀ alkyl, C₁₋₆ haloalkyl, C₂₋₆ alkenyl, or C₂₋₆ alkynyl, wherein said C₁₋₁₀ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy.

In some embodiments, X is absent, C₁₋₁₀ alkyl, C₁₋₆ haloalkyl, or C₂₋₆ alkenyl, wherein said C₁₋₁₀ alkyl, C₁₋₆ haloalkyl, or C₂₋₆ alkenyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy.

In some embodiments, X is absent, C₁₋₁₀ alkyl, C₁₋₆ haloalkyl, or C₂₋₆ alkenyl, wherein said C₁₋₁₀ alkyl, C₁₋₆ haloalkyl, or C₂₋₆ alkenyl is optionally substituted with 1, 2, or 3 substituents independently selected from OH, CN, halo, C₁₋₆ alkyl, C₁₋₆ alkoxy.

In some embodiments, X is absent, C₁₋₁₀ alkyl, or C₂₋₆ alkenyl, wherein said C₁₋₁₀ alkyl or C₂₋₆ alkenyl is optionally substituted with 1, 2, or 3 substituents independently selected from CN, halo, C₁₋₆ alkyl, C₁₋₆ alkoxy.

In various embodiments, X is absent, C₁₋₁₀ alkyl, or C₂₋₆ alkenyl; X is absent or C₁₋₁₀ alkyl; X is absent or CH₂; X is absent; or X is CH₂. In some embodiments, B is absent; or B is a spacer.

In some embodiments, the spacer is an alkyl interrupted by one or more O, NR^{a}, S, SO, SO₂, C(O)O, OC(O), NHCO, CONH, SC(O), or C(O)S, said alkyl is optionally terminated with one or two O, NR^{a}, S, SO, SO₂, C(O)O, OC(O), NHCO, CONH, SC(O), or C(O)S.

In some embodiments, the spacer is a C₁₋₂₀₀ alkyl (e.g., C₁₋₁₅₀ alkyl, C₁₋₁₀₀ alkyl, C₁₋₇₅ alkyl, C₁₋₅₀ alkyl, C₁₋₄₀ alkyl, or C₁₋₂₀ alkyl) interrupted by one or more O, NR^{a}, S, SO, SO₂, C(O)O, OC(O), NHCO, CONH, SC(O), or C(O)S, said alkyl is optionally terminated with one or two O, NR^{a}, S, SO, SO₂, C(O)O, OC(O), NHCO, CONH, SC(O), or C(O)S.

In some embodiments, the spacer includes an alkyl, ether, ester, amide, thioester, thioether, with or without one or more reactive groups such as carboxylic acid, thiol, anhydride, amine, hydroxyl, and/or halogen. In some embodiments, the spacer is functionalized with two or more reactive groups, such that at least one of the reactive groups can conjugate to a particle, and at least one of the remaining reactive groups can conjugate to an ion-chelating molecule via conjugation techniques described, for example, in Hermanson G. T., Bioconjugate Techniques, Academic Press, San Diego, Calif., 1996.

In some embodiments, the spacer is C₁₋₁₄ alkyl, [(C₁₋₁₄ alkyl)S]ₙ, [(C₁₋₁₄ alkyl)OCO]ₙ, [(C₁₋₁₄ alkyl)O]ₙ, [(C₁₋₁₄ alkyl)CONH]ₙ, [(C₁₋₁₄ alkyl)NHCO]ₙ, [S(C₁₋₁₄ alkyl)]ₙ, [OCO(C₁₋₁₄ alkyl)]ₙ, [O(C₁₋₁₄ alkyl)]ₙ, [CONH(C₁₋₁₄ alkyl)]ₙ, or [NHCO(C₁₋₁₄ alkyl)]ₙ, wherein said C₁₋₁₄ alkyl, [(C₁₋₁₄ alkyl)S]ₙ, [(C₁₋₁₄ alkyl)OCO]ₙ, [(C₁₋₁₄ alkyl)O]ₙ, [(C₁₋₁₄ alkyl)CONH]ₙ, [(C₁₋₁₄ alkyl)NHCO]ₙ, [S(C₁₋₁₄ alkyl)]ₙ, [OCO(C₁₋₁₄ alkyl)]ₙ, [O(C₁₋₁₄ alkyl)]ₙ, [CONH(C₁₋₁₄ alkyl)]ₙ, or [NHCO(C₁₋₁₄ alkyl)]ₙ is optionally substituted with 1, 2, 3, 4, 5, or 6 substituents selected from halogen, amino, hydroxyl, thiol, anhydride, and COOH, and n is 1, 2, 3, 4, 5, 6, 7, 8, 9, or 10.

In some embodiments, the spacer is C₁₋₁₄ alkyl, [(C₁₋₁₄ alkyl)OCO]ₙ, [(C₁₋₁₄ alkyl)O]ₙ, [(C₁₋₁₄ alkyl)CONH]ₙ, [(C₁₋₁₄ alkyl)NHCO]ₙ, [OCO(C₁₋₁₄ alkyl)]ₙ, [O(C₁₋₁₄ alkyl)]ₙ, [CONH(C₁₋₁₄ alkyl)]ₙ, or [NHCO(C₁₋₁₄ alkyl)]ₙ, wherein said C₁₋₁₄ alkyl, [(C₁₋₁₄ alkyl)OCO]ₙ, [(C₁₋₁₄ alkyl)O]ₙ, [(C₁₋₁₄ alkyl)CONH]ₙ, [(C₁₋₁₄ alkyl)NHCO]ₙ, [OCO(C₁₋₁₄ alkyl)]ₙ, [O(C₁₋₁₄ alkyl)]ₙ, [CONH(C₁₋₁₄ alkyl)]ₙ, or [NHCO(C₁₋₁₄ alkyl)]ₙ is optionally substituted with 1, 2, 3, 4, 5, or 6 substituents selected from halogen, amino, hydroxyl, thiol, anhydride, and COOH, and n is 1, 2, 3, 4, 5, 6, 7, 8, 9, or 10.

In some embodiments, the spacer is C₁₋₁₄ alkyl, [(C₁₋₁₄ alkyl)OCO]ₙ, [(C₁₋₁₄ alkyl)O]ₙ, [OCO(C₁₋₁₄ alkyl)]ₙ, or [O(C₁₋₁₄ alkyl)]ₙ, wherein said C₁₋₁₄ alkyl, [(C₁₋₁₄ alkyl)OCO]ₙ, [(C₁₋₁₄ alkyl)O]ₙ, [OCO(C₁₋₁₄ alkyl)]ₙ, or [O(C₁₋₁₄ alkyl)]ₙ is optionally substituted with 1, 2, 3, 4, 5, or 6 substituents selected from halogen, amino, hydroxyl, thiol, anhydride, and COOH, and n is 1, 2, 3, 4, 5, 6, 7, 8, 9, or 10.

In some embodiments, the spacer is C₁₋₁₄ alkyl, [(C₁₋₁₄ alkyl)O]ₙ, or [O(C₁₋₁₄ alkyl)]ₙ, wherein said C₁₋₁₄ alkyl, [(C₁₋₁₄ alkyl)O]ₙ, or [O(C₁₋₁₄ alkyl)]ₙ is optionally substituted with 1, 2, 3, 4, 5, or 6 substituents selected from halogen, amino, hydroxyl, thiol, anhydride, and COOH, and n is 1, 2, 3, 4, 5, 6, 7, 8, 9, or 10.

In certain embodiments, n is greater than 10 (e.g., 20, 30, 40, 50, 60, 70, 80, 90, or 100).

In various embodiments, R^{a} is H, C₁₋₁₀ alkyl, C₁₋₆ haloalkyl, C₂₋₆ alkenyl, C₂₋₆ alkynyl, aryl, heteroaryl, cycloalkyl, heterocycloalkyl, or arylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, or arylalkyl, is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy.

In some embodiments, R^{a} is H, C₁₋₁₀ alkyl, C₁₋₆ haloalkyl, C₂₋₆ alkenyl, C₂₋₆ alkynyl, aryl, heteroaryl, cycloalkyl, or heterocycloalkyl, wherein said C₁₋₁₀ alkyl, C₁₋₆ haloalkyl, C₂₋₆ alkenyl, C₂₋₆ alkynyl, aryl, heteroaryl, cycloalkyl, or heterocycloalkyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy.

In other embodiments, R^{a} is H, C₁₋₁₀ alkyl, C₁₋₆ haloalkyl, C₂₋₆ alkenyl, or C₂₋₆ alkynyl, wherein said C₁₋₁₀ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy.

In various embodiments, R^{a} is H, C₁₋₁₀ alkyl, or C₁₋₆ haloalkyl, wherein said C₁₋₁₀ alkyl or C₁₋₆ haloalkyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy.

In some embodiments, R^{a} is H, C₁₋₁₀ alkyl, C₁₋₆ haloalkyl, C₂₋₆ alkenyl, C₂₋₆ alkynyl, aryl, heteroaryl, cycloalkyl, heterocycloalkyl, or arylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, or arylalkyl, is optionally substituted with 1, 2, or 3 substituents independently selected from OH, CN, amino, and halo.

In certain embodiments, R^{a} is H, C₁₋₁₀ alkyl, C₁₋₆ haloalkyl, C₂₋₆ alkenyl, C₂₋₆ alkynyl, aryl, heteroaryl, cycloalkyl, or heterocycloalkyl, wherein said C₁₋₁₀ alkyl, C₁₋₆ haloalkyl, C₂₋₆ alkenyl, C₂₋₆ alkynyl, aryl, heteroaryl, cycloalkyl, or heterocycloalkyl is optionally substituted with 1, 2, or 3 substituents independently selected from OH, CN, amino, and halo.

In some embodiments, R^{a} is H, C₁₋₁₀ alkyl, C₁₋₆ haloalkyl, alkenyl, or C₂₋₆ alkynyl, wherein said C₁₋₁₀ alkyl, C₁₋₆ haloalkyl, C₂₋₆ alkenyl, or C₂₋₆ alkynyl is optionally substituted with 1, 2, or 3 substituents independently selected from OH, CN, amino, and halo.

In various embodiments, R^{a} is H, C₁₋₁₀ alkyl, or C₁₋₆ haloalkyl, wherein said C₁₋₁₀ alkyl or C₁₋₆ haloalkyl is optionally substituted with 1, 2, or 3 substituents independently selected from OH, CN, amino, and halo.

In some embodiments, R^{a} is H, C₁₋₁₀ alkyl, or C₁₋₆ haloalkyl, wherein said C₁₋₁₀ alkyl or C₁₋₆ haloalkyl is optionally substituted with 1, 2, or 3 substituents independently selected from OH and halo.

In certain embodiments, R^{a} is H, C₁₋₁₀ alkyl, or C₁₋₆ haloalkyl, wherein said C₁₋₁₀ alkyl or C₁₋₆ haloalkyl is optionally substituted with 1, 2, or 3 substituents independently selected from OH and halo. In some embodiments, R^{a} is H, C₁₋₁₀ alkyl, or C₁₋₆ haloalkyl, wherein said C₁₋₁₀ alkyl or C₁₋₆ haloalkyl is optionally substituted with 1, 2, or 3 halo. In other embodiments, R^{a} is H, C₁₋₁₀ alkyl, or C₁₋₆ haloalkyl; or R^{a} is H or C₁₋₁₀ alkyl.

In some embodiments, c is 0, and in certain embodiments, a and b are each independently 0 or 1, c is 0, and a+b+c is greater than or equal to 1.

The new compounds and/or particles described herein are designed to be stable.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

It is understood that when a substituent is depicted structurally as a linking moiety, it is necessarily minimally divalent. For example, when the variable B of the structure depicted in Formula I is alkyl, the alkyl moiety is understood to be an alkyl linking moiety such as —CH₂—, —CH₂CH₂—, CH₃CH<, etc.

It is intended that when a is 0, Y is bound to particle M via -(D-B—X-A)_{b}- and -(D-B—X-A)_{c}—, i.e.,

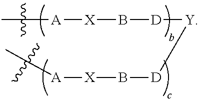

It is intended that when b is 0, Y is bound to particle M via -(D-B—X-A)ₐ- and -(D-B—X-A)_{c}-, i.e.,

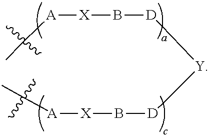

It is intended that when c is 0, Y is bound to particle M via -(D-B—X-A)ₐ- and -(D-B—X-A)_{b}-, i.e.,

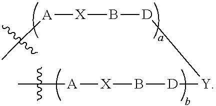

Similarly, it is intended that when any two of a, b, and c are 0, Y is bound to particle M via -(D-B—X-A)-, i.e.,

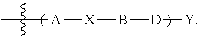

As used herein, the term “spacer” refers to a chemical linker situated between the ion-chelating molecule and the magnetic particle. The spacer is linked to the ion-chelating molecule and the magnetic particle via connecting covalent linkages such as NHCO, CONH, S, O, or NR^{a}. In some embodiments, the linker is separated from the connecting covalent linkages via a moiety X.

As used herein, the term “alkyl” refers to a saturated hydrocarbon group which is straight-chained or branched. Examples of alkyl groups include methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like. As an example, an alkyl group can contain from 1 to 20, from 2 to 20, from 1 to 14, from 1 to 10, from 1 to 8, from 1 to 6, from 1 to 4, or from 1 to 3 carbon atoms.

As used herein, the term “alkyl interrupted by one or more” denotes straight chain or branched alkyl e.g. C₁₋₂₀₀ alkyl, in which one or more pairs of carbon atoms are linked by O, NR^{a}, S, SO, SO₂, C(O)O, OC(O), NHCO, CONH, SC(O), or C(O)S.

As used herein, “alkenyl” refers to an alkyl group having one or more double carbon-carbon bonds. Example alkenyl groups include ethenyl, propenyl, and the like.

As used herein, “alkynyl” refers to an alkyl group having one or more triple carbon-carbon bonds. Example alkynyl groups include ethynyl, propynyl, and the like.

As used herein, “haloalkyl” refers to an alkyl group having one or more halogen substituents. Example haloalkyl groups include CF₃, C₂F₅, CHF₂, CCl₃, CHCl₂, C₂Cl₅, and the like.

As used herein, “aryl” refers to monocyclic or polycyclic (e.g., having 2, 3 or 4 fused rings) aromatic hydrocarbons such as, for example, phenyl, naphthyl, anthracenyl, phenanthrenyl, indanyl, indenyl, and the like. In some embodiments, aryl groups have from 6 to about 20 carbon atoms.

As used herein, “cycloalkyl” refers to non-aromatic carbocycles including cyclized alkyl, alkenyl, and alkynyl groups. Cycloalkyl groups can include mono- or polycyclic (e.g., having 2, 3 or 4 fused rings) ring systems, including spirocycles. In some embodiments, cycloalkyl groups can have from 3 to about 20 carbon atoms, 3 to about 14 carbon atoms, 3 to about 10 carbon atoms, or 3 to 7 carbon atoms. Cycloalkyl groups can further have 0, 1, 2, or 3 double bonds and/or 0, 1, or 2 triple bonds. Also included in the definition of cycloalkyl are moieties that have one or more aromatic rings fused (i.e., having a bond in common with) to the cycloalkyl ring, for example, benzo derivatives of pentane, pentene, hexane, and the like. A cycloalkyl group having one or more fused aromatic rings can be attached through either the aromatic or non-aromatic portion. One or more ring-forming carbon atoms of a cycloalkyl group can be oxidized, for example, having an oxo or sulfido substituent. Example cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcamyl, adamantyl, and the like.

As used herein, a “heteroaryl” group refers to an aromatic heterocycle having at least one heteroatom ring member such as sulfur, oxygen, or nitrogen. Heteroaryl groups include monocyclic and polycyclic (e.g., having 2, 3 or 4 fused rings) systems. Any ring-forming N atom in a heteroaryl group can also be oxidized to form an N-oxo moiety. Examples of heteroaryl groups include without limitation, pyridyl, N-oxopyridyl, pyrimidinyl, pyrazinyl, pyridazinyl, triazinyl, furyl, quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like. In some embodiments, the heteroaryl group has from 1 to about 20 carbon atoms, and in further embodiments from about 3 to about 20 carbon atoms. In some embodiments, the heteroaryl group contains 3 to about 14, 3 to about 7, or 5 to 6 ring-forming atoms. In some embodiments, the heteroaryl group has 1 to about 4, 1 to about 3, or 1 to 2 heteroatoms.

As used herein, “heterocycloalkyl” refers to a non-aromatic heterocycle where one or more of the ring-forming atoms is a heteroatom such as an O, N, or S atom. Heterocycloalkyl groups can include mono- or polycyclic (e.g., having 2, 3 or 4 fused rings) ring systems as well as spirocycles. Example “heterocycloalkyl” groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles such as indolene and isoindolene groups. A heterocycloalkyl group having one or more fused aromatic rings can be attached though either the aromatic or non-aromatic portion. In some embodiments, the carbon atoms or heteroatoms in the heterocyclyl or heterocycle ring can be oxidized (to form, e.g., a carbonyl, sulfinyl, sulfonyl, or other oxidized nitrogen or sulfur linkage) or a nitrogen atom can be quaternized. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains 0 to 3 double bonds. In some embodiments, the heterocycloalkyl group contains 0 to 2 triple bonds.

As used herein, “halo” or “halogen” includes fluoro, chloro, bromo, and iodo.

As used herein, “alkoxy” refers to an —O-alkyl group. Example alkoxy groups include methoxy, ethoxy, propoxy (e.g., n-propoxy and isopropoxy), t-butoxy, and the like.

As used herein, “haloalkoxy” refers to an —O-(haloalkyl) group.

As used herein, “arylalkyl” refers to alkyl substituted by aryl and “cycloalkylalkyl” refers to alkyl substituted by cycloalkyl. An example arylalkyl group is benzyl.

As used herein, “heteroarylalkyl” refers to alkyl substituted by heteroaryl and “heterocycloalkylalkyl” refers to alkyl substituted by heterocycloalkyl.

As used herein, “amino” refers to NH₂.

As used herein, “dialkylamino” refers to an amino group substituted by two alkyl groups.

As used herein, “halogen” includes fluoro, chloro, bromo, and iodo.

### Magnetic Particles

Particles that can be used in the ion-binding assays described herein include magnetic metal oxides (e.g., iron oxide or magnetite), such as cross-linked iron oxides and/or monocrystalline iron oxides. The metal oxides can be present in the particles in a core, or as a shell over a polymer core. The magnetic metal oxides can also include cobalt, magnesium, zinc, or mixtures of these metals with iron. The particles can have a substantially spherical shape and defined surface chemistry so as to decrease chemical agglutination and non-specific binding. In some embodiments, the particles are irregularly shaped. The particles can have a surface coating such as a polymer (e.g., polystyrene, polyethylene glycol, dextran) to encase the iron oxide and decrease the exposure of the test sample to the metallic core. In some embodiments, the particles are coated with saccharide, polysaccharide (e.g., dextran), or a chemical substance having a single type of functional groups. In some embodiments, the particles are functionalized with reactive groups such as amino, carboxylic acid, hydroxyl, thiol, anhydride, or halogen to react with linkers and/or ion-chelating molecules. When the particles aggregate in an aqueous environment, they can cause a detectable change in relaxation properties (e.g., the T2 relaxation) of the surrounding solvent (e.g., water).

Table 1 shows a variety of magnetic particles that can be used to prepare the new ion-binding particles.

TABLE 1
Examples of particles for preparing
ion-binding magnetic particles

| Number or Entry | Particle | Supplier/Origin |
|---|---|---|
| 1 | MION or CLIO | Center for Molecular |
| | | Imaging Research, |
| | | Charlestown MA |
| 2 | MyOne ™ | Dynal/Invitrogen, Carlsbad |
| | | CA |
| 3 | MACS | Miltenyi Biotech, |
| | | Auburn, CA |
| 4 | Magnetic particles | G. Kisker GbR, Steinfurt |
| | | Germany |
| 5 | MagNa Gel | Alnis Biosciences, Research |
| | | Triangle Park, NC |
| 6 | Magnetic Particles | Spherotech Inc., Lake |
| | | Forest IL |
| 7 | Magnetic Spheres | Polysciences Inc., |
| | | Warrington PA |
| 8 | Microspheres | Estapor Microsphere, |
| | | Fontenay Sous Bois, France |

The particles can be uniform or non-uniform in size. In some embodiments, the particles have a maximum dimension of more than 10 nm (e.g., more than 20 nm, more than 40 nm, more than 60 nm, more than 80 nm, more than 100 nm, more than 200 nm, more than 400 nm, more than 600 nm, more than 800 nm, more than one micron, more than 1.5 microns, more than two microns, more than five microns, more than 10 microns, more than 25 microns, more than 50 microns, more than 75 microns) and/or less than or equal to 100 microns (e.g., less than 75 microns, less than 50 microns, less than 25 microns, less than 10 microns, less than five microns, less than two microns, less than 1.5 microns, less than one micron, less than 800 nm, less than 600 nm, less than 400 nm, less than 200 nm, less than 100 nm, less than 80 nm, less than 60 nm, less than 40 nm, or less than 20 nm). In some embodiments, the particles can have a maximum dimension of from 10 nm to 200 nm (e.g., 10 nm to 100 nm, 20 nm to 100 nm, 40 nm to 100 nm, 40 to 200 nm, from 200 nm to 500 nm, from 200 mu to one micron, from 500 nm to two microns, from one to two microns, from one to five microns, from one to 20 microns, from 100 nm to 100 microns).

When suspended in a solution, the magnetic particles are non-settling (i.e., the particles remain essentially suspended) in the liquid sample for extended periods of time. As used herein, the term “non-settling” refers to particles having a relatively low tendency to settle by gravity during the course of the assay (i.e., particles that when in a collection, remain essentially suspended, as defined herein, in the liquid sample during the course of the assay). Candidate non-settling particles are evaluated using conventional light scattering techniques. A suspension containing the candidate particles and a solvent or a medium used to actually test the particles in later assays (total volume of 0.4 milliliters (mL)) is introduced into a 1 mL cuvette (the sample and cuvette volumes are chosen so as to create a relatively flat sample, thereby maximizing contact of the entire height of the sample with the light source). The cuvette is then placed in a light scattering machine (e.g., by Malvern Instruments, Southborough, Mass.), and the optical density of the suspension is monitored over a 2 hour period at room temperature. Particles that exhibit less than a 10% change in optical density are “non-settling” and thus suitable for use in the methods described herein.

As used herein, the term “magnetic particles” refers to any particle that is always magnetic and any particle that has a magnetic moment under certain conditions (e.g., in an applied electromagnetic field). Particle settling can generally be avoided by using relatively small particles (e.g., particles) or relatively large particles whose density is comparable to that of water. The density of particles can be altered by using polymers of different densities in their synthesis. In all embodiments, the particles have a surface that permits the attachment of biological molecules.

In general, the particles can have a relatively high relaxivity owing to the superparamagnetism of their iron or metal oxide. In some embodiments, the particles have an RI relaxivity between about 5 and 30 mM⁻¹ sec⁻¹, e.g., 10, 15, 20, or 25 mM⁻¹ sec⁻¹. In some embodiments, the particles have an R2 relaxivity between about 15 and 100 mM⁻¹ sec⁻¹, e.g., 25, 50, 75, or 90 mM⁻¹ sec⁻¹. In some embodiments, particles have a ratio of R2 to R1 of between 1.5 and 4, e.g., 2, 2.5, or 3. In some embodiments, the particles have an iron oxide content that is greater than about 10% of the total mass of the particle, e.g., greater than 15, 20, 25 or 30 percent.

In some embodiments, when the magnetic particle is an iron oxide-based particle, concentrations of iron (Fe) in a sample can be from about 2 micrograms (μg)/mL to about 50 μg/mL Fe. In general, the iron concentration is selected so as to be sufficiently high to alter the relaxation properties of water. For particles with relatively high relaxivities, lower iron concentrations can be used. For particles with relatively low relaxivities, higher iron concentrations can be used.

### Methods of Making Particles

There are a variety of ways that the particles can be prepared, but in all methods, the result must be a particle with one or more functional groups that can be used to link the particle to the ion-chelating molecule.

For example, non-polymeric surface-functionalized metal oxide particles can be synthesized according to the method of Albrecht et al., Biochimie, 80 (5-6): 379-90, 1998. Dimercapto-succinic acid is coupled to the iron oxide and provides a carboxyl functional group. By functionalized is meant the presence of amino or carboxyl or other reactive groups.

There are several methods for synthesizing carboxy- and amino-derivatized particles. Carboxy-functionalized particles can be made, for example, according to the method of Gorman (see WO 00/61191). In this method, reduced carboxymethyl (CM) dextran is synthesized from commercial dextran. The CM-dextran and iron salts are mixed together and are then neutralized with ammonium hydroxide. The resulting carboxy-functionalized particles can be used for coupling to ion-chelating molecules. Carboxy-functionalized particles can also be made from polysaccharide coated particles by reaction with bromo or chloroacetic acid in strong base to attach carboxyl groups. In addition, carboxy-functionalized particles can be made from amino-functionalized particles by converting amino to carboxy groups by the use of reagents such as succinic anhydride or maleic anhydride.

Particle size can be controlled by adjusting reaction conditions, for example, by using low temperature during the neutralization of iron salts with a base as described in U.S. Pat. No. 5,262,176. Uniform particle size materials can also be made by fractionating the particles using centrifugation, ultrafiltration, or gel filtration, as described, for example in U.S. Pat. No. 5,492,814.

Particles can also be synthesized according to the method of Molday (Molday, R. S. and D. MacKenzie, “Immunospecific ferromagnetic iron-dextran reagents for the labeling and magnetic separation of cells,” J. Immunol. Methods, 1982, 52(3):353-67, and treated with periodate to form aldehyde groups. The aldehyde-containing particles can then be reacted with a diamine (e.g., ethylene diamine or hexanediamine), which will form a Schiff base, followed by reduction with sodium borohydride or sodium cyanoborohydride.

Dextran-coated particles can be made and cross-linked with epichlorohydrin. The addition of ammonia will react with epoxy groups to generate amine groups, see Hogemann, D., et al., Improvement of MRI probes to allow efficient detection of gene expression Bioconjug. Chem., 2000, 11(6):941-6, and Josephson et al., “High-efficiency intracellular magnetic labeling with novel superparamagnetic-Tat peptide conjugates,” Bioconjug. Chem., 1999, 10(2):186-91. This material is known as cross-linked iron oxide or “CLIO” and when functionalized with amine is referred to as amine-CLIO or NH₂-CLIO.

Carboxy-functionalized particles can be converted to amino-functionalized magnetic particles by the use of water-soluble carbodiimides and diamines such as ethylene diamine or hexane diamine.

Avidin or streptavidin can be attached to particles for use with a biotinylated ion-chelating molecule. See e.g., Shen et al., “Magnetically labeled secretin retains receptor affinity to pancreas acinar cells,” Bioconjug. Chem., 1996, 7(3):311-6. Similarly, biotin can be attached to a particle for use with an avidin-labeled binding moiety.

In all of these methods, low molecular weight compounds can be separated from the particles by ultra-filtration, dialysis, magnetic separation, or other means. The unreacted oligonucleotides can be separated from the oligonucleotide-particle conjugates, e.g., by magnetic separation or size exclusion chromatography.

### Ion-Chelating Molecules

In some embodiments, a useful ion-chelating molecule is a small molecule (e.g., a molecule having a molecular weight less than or equal to 1,000). The ion-chelating molecule can include a reactive group such as amino, carboxylic acid, thiol, anhydride, hydroxyl, or halogen to covalently bond to a particle via a chemical reaction. Two or more ion-chelating molecule can complex to the same ionic analyte to form an aggregate structure, for example, via hydrogen bonds, ionic bonds, or donor-acceptor bonds.

Table 2 shows selected examples of ion-chelating molecules, e.g., synthetic molecules, that can be used to prepare the new ion-binding particles. The synthetic ion-chelating molecules can be selected from large libraries of ion carriers (or ionophores) developed for ion selective electrodes. For example, proposed ion-chelating molecules in example entries 1, 2, 5, 8, 10, 11, and 12 in Table 2 were adapted from ionophores available from the Aldrich catalogue. Example entries 3, 4, 6, 7, 9, and 13 were adapted from other sources of ion carriers developed for ion-selective electrodes. Although libraries of chelators developed for ion-selective electrodes are the largest known and therefore are the primary source of chelating molecules for the new NMR-based methods of measuring ions using magnetic particles, synthetic ion chelating molecules developed for other applications such as MRI agents or other particle-based ion sensors can also be used (See, e.g., example entries 11, 12, and 14). It should be noted that synthetic ion chelating molecules used in turbidity, fluorimetry, colorimetry, or other assays based on chelation to ions could in some cases also be adapted to the new methods although, in practice, these chelating molecules are often themselves derived from ion carriers used in ion-selective electrodes.

TABLE 2
Selected ion-chelating molecules for ion-binding magnetic particles

| Entry, Scheme, Example | Chelating molecule | Chelated Ion |
|---|---|---|
| 1, Sch 1, Ex. 1 | 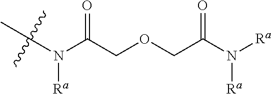 | calcium |
| 2, Sch 2, Ex. 2 | 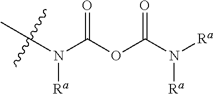 | magnesium |
| 3 | 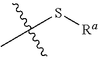 | silver |
| 4 | 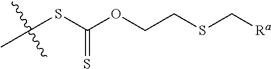 | samarium |
| 5 | 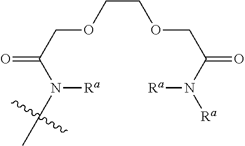 | lead |
| 6, Sch 6 Ex. 6 | 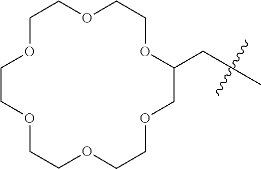 | cesium |
| 7 | 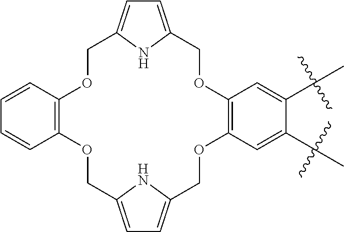 | ammonium |
| 8, Sch 3, Ex. 3 | 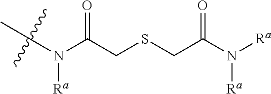 | copper |
| 9 | 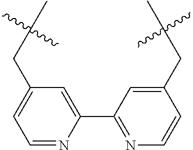 | cadmium |
| 10 | 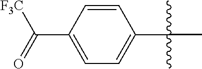 | carbonate |
| 11, Sch 4, Ex. 4 | 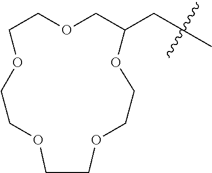 | potassium |
| 12, Sch 5, Ex. 5 | 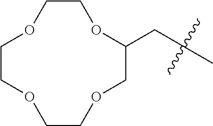 | sodium |
| 13 | 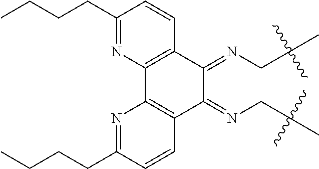 | lithium |
| 14, Sch 7, Ex. 7 | 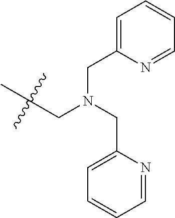 | zinc |

R^{a} can be the same or different on any given ion-chelating molecule, and is as defined above.

The ability of example entries 1-14 to selectively recognize ions has been demonstrated, for example, in Suzuki et al., Analytical Chemistry, 1995, 67, 324-334 for entries 1-2; Hisamoto et al., Analytica Chimica Acta, 1994, 299, 179-187 for entry 1; Buhlmann et al., Chemical Reviews, 1998, 98, 1593-1687 for entries 1-6 and 9-13; Aldrich Chemical Company for entries 1-2, 5, 8, and 10-12; Odonnell et al., Chimica Acta, 1993, 281, 129-134, Eugster et al., Clinical Chemistry, 1993, 39, 855-859, Hu et al., Analytical Chemistry, 1989, 61, 574-576, and Erne et al., Helvetica Chimica Acta, 1980, 63, 2271-2279 for entry 2; Casabo et al., Inorganic Chemistry, 1995, 34, 5410-5415, Errachid et al., Sensors and Actuators B-Chemical, 1995, 27, 321-324, and Teixidor et al., Journal of the Chemical Society—Chemical Communications, 1994, 963-964 for entry 3; Chowdhury et al., Analytical Chemistry, 1996, 68, 366-370 for entry 4; Lerchi et al., Analytical Chemistry, 1992, 64, 1534-1540, and Malinowska, E. Analyst, 1990, 115, 1085-1087 for entry 5; Kimura et al., Journal of the Chemical Society—Perkin Transactions 2, 1984, 447-450 for entries 6, 11, and 12; Kimura et al., Journal of Electroanalytical Chemistry, 1979, 105, 335-340 for entry 6; Kariuki et al., Crystal Growth & Design, 2002, 2, 309-311 for entry 7; Szigeti et al., Analytica Chimica Acta 2005, 532, 129-136 for entry 8; Stevens et al., Analytica Chimica Acta, 1991, 248, 315-321 for entry 9; Behringer et al., Analytica Chimica Acta, 1990, 233, 41-47, and Meyerhoff et al., Analytical Chemistry, 1987, 59, 144-150 for entry 10; Kimura et al., Journal of Electroanalytical Chemistry, 1979, 95, 91-101, Kimura et al., Journal of the Chemical Society—Chemical Communications, 1983, 492-493, Moody et al., Analyst, 1989, 114, 15-20, Kim et al., Angewandte Chemie—International Edition, 2000, 39, 3868-3871, Chen et al., Chemical Communications, 2006, 263-265, and Lin et al., Analytical Chemistry, 2002, 74, 330-335 for entry 11; Tamura et al., Analytical Chemistry, 1982, 54, 1224-1227, Lin et al., Analytical Chemistry, 2005, 77, 4821-4828, and Flink et al., Journal of the American Chemical Society, 1998, 120, 4652-4657 for entries 11 and 12; Obare et al., Langmuir, 2002, 18, 10407-10410, Sugihara et al., Coordination Chemistry Reviews, 1996, 148, 285-299 for entry 13; Trokowski et al., Angewandte Chemie—International Edition, 2005, 44, 6920-6923, Hanaoka et al., Journal of the Chemical Society—Perkin Transactions 2, 2001, 1840-1843, Hanaoka et al., Chemistry & Biology, 2002, 9, 1027-1032 14, and Quinti et al., Nano Letters, 2006, 6, 488-490 for entry 14.

### Methods of Making Ion-Binding Particles

Once an appropriate magnetic particle and a specific ion-chelating molecule have been selected, they are linked, e.g., using the following steps. An important point in selecting the proper chelating molecule-particle motif for NMR-based methods of measuring ions using magnetic particles is to select motifs that will coordinate to the ion such that complexes formed around the target ion have two or more chelating molecules to bring the magnetic particles together. Introduction of a reactive group to the chelating molecule structure or the use of chelating molecule precursors is sometimes required for conjugation to the particles as described in the examples below. Moreover, the use of chemical spacers added to the chelator structure and the use of additional ligands on the magnetic particle surface can improve the detection of ions in some cases.

Conjugation of the ion-chelating molecule to a particle can occur via a covalent linkage. For example, and amine moiety can react with a carboxylic acid using a coupling agent (e.g., a carbodiimide, such as 1-ethyl-3-[3-dimethylaminopropyl]carbodiimide hydrochloride, or N,N′-dicyclohexyl-carbodiimide) to form an amide linkage, a hydroxyl moiety can react with a halogen group to form an ether linkage, a hydroxyl moiety can react with a carboxylic acid moiety using a coupling agent to form an ester linkage. Conjugation techniques are described in detail, for example, in Hermanson G. T., Bioconjugate Techniques, Academic Press, San Diego, Calif., 1996.

In some embodiments, the ion-chelating particle includes a chemical spacer between the particle and the ion-chelating molecule. As an example, a spacer can include an alkyl, ether, ester, amide, thioester, thioether, with or without one or more reactive groups such as carboxylic acid, thiol, anhydride, amine, hydroxyl, and/or halogen. In some embodiments, the spacer is functionalized with two or more reactive groups, such that at least one of the reactive groups can covalently bind to a particle, and at least one of the remaining reactive groups can covalently bind to an ion-chelating molecule via conjugation techniques described, for example, in Hermanson Bioconjugate Techniques, Academic Press, San Diego, Calif., 1996.

In some embodiments, an ion-binding magnetic particle can contain one or more ion-chelating molecules (e.g., 2, 4, 6, 8, 10, 15, 20, 25, 30, 35, 40, 45, 50, 60, 70, 80, 90, 100, 200, 300, 400, 500, 600, 700, 800, 900, or 1000 ion-chelating molecules), depending on the size of the particle. For example, a microparticle having a maximum dimension of equal or larger than one micron can include a larger number of ion-chelating molecules, e.g., 200 to 1000, 200 to 800, 200 to 600, or 200 to 600 ion-chelating molecules. A nanoparticle having a maximum dimension of less than one micron can have a smaller number of ion-chelating molecules, e.g., 2 to 200, 2 to 150, 2 to 100, or 2 to 50 ion-chelating molecules. The number of ion-chelating molecules per particle can vary within a given population of particles. The number of ion-chelating molecules per particle can be assessed using well-known titration methods or protein chemistry methods. In some embodiments, one or more different types of ion-chelating molecules can be bound to the same particle, and/or a population of particles can contain one or more types of particles capable of binding to one or more types of ionic analyte.

### Methods of Measuring Ion Concentrations in Samples

The new methods can employ ion-binding magnetic particles in assays by suspending them in a sample to detect the presence of specific ions in a sample, or to measure the concentration of specific ions in the sample (assay format). In this format there is no surface or electrode to foul, which is a major issue with current electrode based ion sensors. The current invention also includes semi-permeable devices that enclose and retain the ion binding magnetic particles, but enable ions to enter. Here the ion binding particles are a component of a continuous sensor as described by Sun et al., in U.S. application Ser. No. 11/431,247. In both assay and sensor formats, particle-based ion assays overcome one of the main limitations of ion selective electrode methods, which is a short lifetime of the electrode due to clogging of the membrane.

The ion-binding particles can be used in a wide variety of medical and industrial applications. For example, the ion-binding particles can be used for testing for the presence and/or concentration of specific ions in samples such as biological fluids (e.g., blood, serum, urine, interstitial fluid, or cerebral spinal fluid, of a human or animal subject). In some embodiments, the ion-binding particles are used in industrial applications, e.g., drinking water monitoring, wastewater treatment, chemical processing, preparations of medical and industrial buffers and solutions, food processing, and/or paper manufacture.

In some embodiments, an assay format includes addition of an analyte ion to a suspension of ion-binding magnetic particles. A change in relaxation time can occur due to clustering of the particles around the ionic analyte by chelation of at least two ion-chelating molecules. The change in relaxation time is indicative of the presence of the analyte and the amount of change in relaxation time can be correlated with the concentration of the ionic analyte, for example, by comparing the change in relaxation time with a calibration equation/curve for a series of different concentrations of an ionic analyte and/or a standard. In some embodiments, the rate of change in relaxation time can be correlated with the concentration of the ionic analyte. The sensitivity of the assay can relate to the concentration of the magnetic particles. By changing the particle concentration, the sensor could be tuned to the region of interest for the detection of a particular ionic analyte. In some embodiments, the difference in relaxation time can correspond to the difference in relaxation time of a suspension of ion-binding magnetic particles without ions and the relaxation time of a separate but identical suspension with ions. In some embodiments, the assay is reversible, for example, by adding a competing chelating molecule (e.g., EDTA, EGTA, DTPA, NTA acid, o-phenanthroline, dimercaptopropanol, and/or salicylic acid) for the ionic analyte to regenerate the ion-chelating molecules on the magnetic particles.

In other embodiments, an assay format includes obtaining a sample including an ionic analyte (e.g., an ionic analyte that is available for binding to an ion-binding magnetic particle), contacting the sample with one or more ion-binding magnetic particles such that complexes of the ionic analyte and the ion-binding magnetic particles can form, and measuring the relaxation time of the sample. The relaxation time of the sample can be compared with the relaxation time of a reference. The difference in relaxation time (or rate of change of the relaxation time) of the sample and the relaxation time (or rate of change of the relaxation time) of the reference can indicate the presence and/or concentration of the ionic analyte.

In some embodiments, the reference is a separate portion of the sample that is free of ionic analyte (e.g., an ionic analyte available for binding to an ion-binding magnetic particle) and serves as a control sample. For example, the separate portion of the sample can be exposed to a chelating molecule or agent that binds to all or substantially all of the ionic analytes in the sample and prevents them from interacting with the ion-binding magnetic particles. As another example, the reference can be a separate portion of the sample where the ionic analyte is physically removed from the sample, e.g., by dialysis. The relaxation property of the reference can be obtained by contacting the reference with one or more ion-binding magnetic particles, and measuring the relaxation time of the reference.

In some embodiments, the reference is a separate portion of the sample including the ionic analyte. This sample reference can serve as a control by being contacted with non-ion-binding magnetic particles that have the same or similar compositions as the ion-binding particles (e.g., they are the same magnetic particles M), but that are free of ion-chelating molecules Y. The relaxation property of this control sample reference can be obtained by contacting the reference with one or more non-ion-binding magnetic particles, and measuring the relaxation time of the reference.

In other embodiments, the relaxation property of the sample is converted into data. The relaxation properties of a series of reference or control samples can be also converted into data, which can be in the form of a calibration curve, a database, and/or a library. By comparing, e.g., automatically or electronically comparing, the data of relaxation property of the sample with the data of the reference (e.g., calibration data), the difference in relaxation times of the sample and the reference can indicate the presence and/or concentration of the ionic analyte. In some embodiments, when the calibration curve, database, and/or library of the relaxation properties of the reference samples include a sufficient amount of information, the presence and/or concentration of the ionic analyte in a given sample can be directly obtained by comparing with the calibration curve, database, and/or library without the need for a control sample.

In some embodiments, the presence of protons and other ions can be detected via indirect measurement methods. For example, in some embodiments, the ion-binding magnetic particles can bind to a secondary ion different from an ionic analyte (e.g., protons), and the ionic analyte can mediate the binding of a secondary ion and/or compete with the secondary ion for binding to the ion-binding magnetic particles. For example, in some embodiments, when a proton is the ionic analyte, a secondary ion can be kept at a constant concentration, and by changing the proton concentration, any change in the relaxation properties of the solvent due to variable binding of the secondary ion can be correlated with the proton concentration. Indirect methods for measuring ion concentrations are described, for example, in Lauble M. W. et al., Analytical Chemistry, 1985, 57, 2756-2758.

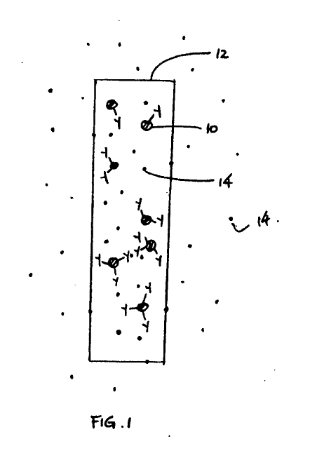

In other embodiments, the ion-binding magnetic particles are contained in a semi-permeable device, as described, for example, in U.S. Ser. No. 11/431,247, and in Sun et al., Small, 2006, 2(10), 1144-1147, and shown in FIG. 1. As shown in FIG. 1, the particles 10 are encapsulated within a semi-permeable walled enclosure 12, e.g., an enclosure that retains the particles, but allows for passage of the ionic analyte 14 into and out of the confines of a sensor chamber. The walled enclosure can have one or more openings sized to enable the passage of the analyte, but not the particles.

When the analyte is absent, the particles are non-aggregated. Upon binding of an analyte, the particles become aggregated. The presence and quantity of the exogenous analyte can be sensed, for example, as a change in the T2 relaxation times of water inside of the sensor chamber. It is known, for example, that water T2 relaxation times shorten upon aggregation or clustering of previously dispersed (e.g., monodispersed, polydispersed) magnetic particles. While not wishing to be bound by theory, it is believed that during particle self-assembly into higher order assemblies, the superparamagnetic iron oxide core of individual particles becomes more efficient at dephasing the spins of the surrounding water protons (i.e., enhancing spin-spin relaxation times, e.g., T2 relaxation times).

Thus, in some embodiments, the analyte can be detected and quantified in the sampling media by monitoring the relaxation properties of the water that is present within the sensor chamber (e.g., measuring changes, such as increases and decreases in T2 relaxation times of water that is present within the sensor chamber). For example, the T2 relaxation times of the water inside of the sensor chamber can decrease in the presence of analyte (due to formation of the particle aggregates) and can increase relative to these depressed values in the absence of analytes (due to displacement of the analytes and subsequent deaggregation of the bound particles). Since the particles are confined within the sensor chamber, the changes in particle aggregation occurring within the sensor chamber in general do not substantially alter the proton relaxation of water outside of the chamber (i.e., bulk water).

Although measuring T2 can be a desirable method for determining particle aggregation, any water relaxation phenomena associated with particles or with their change in aggregation state can be used. T2 can generally be determined in a relatively fast and facile manner. However, measurements of particle aggregation can use T2 in conjunction with other relaxation processes such as T1. In some embodiments, measurements of T1 can be used to correct for small changes in particle concentration within the sensor, due to a small expansion of contraction of the chamber and/or of the particles.

In some embodiments, the semipermeable sensors can be, for example, tubular, spherical, cylindrical, or oval shaped. The sensors described herein can have other shapes as well.

In some embodiments, the size and shape of the sensor can be selected to accommodate a desired or convenient sample holder size and/or sample volume (e.g., in in vitro sensing applications). In general, the volume of the sensor can be selected to enable the sensor to distinguish between the relaxation properties of water inside of the chamber and the water outside of the chamber. For example, the sensor size can be selected so as to accommodate a sample volume of from about 0.1 microliters (μL) to about 1000 milliliters (mL) (e.g., about 1 μL (e.g., with animal imagers), 10 μL (e.g., with clinical MRI instruments) or 0.5 mL. In certain embodiments, the sensor can have a tubular shape in which the open end of the tube has a diameter of from about 1 millimeter (mm) to about 10 mm (e.g., 5 mm 7.5 mm).

In some embodiments, the sensor size and shape can be selected on the basis of the spatial resolution capabilities of conventional magnetic resonance technology (e.g., in in vitro sensing applications). In certain embodiments, the longest dimension of the sensor can be from about 0.01 mm to about 2 mm (e.g., 0.05, 0.1, 0.2, 0.3, 0.4, 0.5, 0.6, 0.7, 0.8, 0.9, 1). In certain embodiments, the applied magnetic field can be, for example, about 0.47 Tesla (T), 1.5 T, 3 T, or 9.4 T (animal assays generally).

In general, the walled enclosure can be any semipermeable material (e.g., a biocompatible semipermeable material) that is permeable to the exogenous analyte and water and substantially impermeable to the particles and the binding agent. In some embodiments, the semipermeable material can be an ultrafiltration or dialysis membrane. In some embodiments, the semipermeable material can be a polymeric substance (e.g., polymeric substances used for encapsulating transplanted cells, see, e.g., M. S. Lesney, Modern Drug Discovery, 2001, 4, 45). In some embodiments, the semipermeable material can be a material used in small implantable, sustained release devices (e.g., those used in implantable, sustained release birth control devices, e.g., Depo-Provera, Norplant, Progestasert; or those described in C. I. Thompson et al., Can. J. Physiol. Pharmacol., 80, 180-92 (March, 2002) or D. C. Stoller, S. R. Thornton, F. L. Smith, Pharmacology, 66, 11-8 (September, 2002)).

The walled enclosure are relatively resistant to fouling or coating under the sampling conditions, thereby increasing the likelihood that the walled enclosure can maintain the specified pore size of the openings (e.g., increasing the likelihood that openings will remain substantially unblocked during sensing). Fouling is the closure of pores (e.g., openings) due to the adsorption of protein that blocks the pores. Fouling can be ascertained by placing materials in biological fluids (e.g., blood) and evaluating their performance using biocompatibility testing methods known in the art.

In some embodiments, the walled enclosure can be designed to be essentially nonimmunogenic, thereby minimizing the likelihood of causing unwanted immune or toxic side effects in a subject (e.g., a human). The walled enclosure can be engineered to be non-immunogenic by using known non-immunogenic materials, such as polyethylene glycol, silicone, poly(DL-lactide-ε-caprolactone), polylactic acid, and/or polyglycolide.

Examples of biocompatible, semipermeable materials include without limitation polysaccharide based materials (cellulose), modified carbohydrate (cellulose ester), or polyvinyl pyrolidine.

In some embodiments, the walled enclosure can be made of a relatively inflexible semipermeable material, such that the encapsulated sensor chamber is a true space or void that does not substantially change in volume when contacted with the fluid sample media. In other embodiments, the walled enclosure can be a relatively flexible semipermeable material, meaning, for example, that the encapsulated sensor chamber can expand in volume when contacted with the fluid sample media (e.g., by intake of the fluid sample media).

In general, the walls of the enclosure are sufficiently thin to allow rapid sensor equilibration to changes in exogenous analyte levels. In some embodiments, the membrane that forms the wall can have a thickness of from about 1 and about 500 hundred microns.

In some embodiments, molecular exclusion can be exclusion by molecular weight. In certain embodiments, each of the openings in the walled enclosure can have a pore size of greater than or equal to 1000 Da. The pore size must be larger than the ionic analyte molecules and smaller than the magnetic particles, such that the ionic analytes have free passage through the enclosure while sequestering the magnetic particles. For example, the pore size can be ½ micron in maximum dimension when the magnetic particles are one micron or larger.

In certain embodiments, the semipermeable material can be cellulose-based materials such as those found in Spectra/Por® tubing, Slide-A-Lyzer® microcassettes, or dialysis fibers. Such materials are generally preferred for applications not involving implantation. In general, the semipermeable material has a pore size that is larger in size than the analyte to permit passage of the analyte into and out of the chamber, but sufficiently small to retain magnetic particles and other reagents such as binding agents (e.g., a binding protein) within the confines of the chamber. The semipermeable material can be selected for the stability (long term function) in the fluid, which contains the analyte to be measured (e.g., blood plasma, interstitial fluid, cerebral spinal fluid of a human or animal subject). The semipermeable material can be further selected on the basis of whether the sensor is implanted or whether the fluid to be assayed is contained within a vessel that is outside of the subject (e.g., a bioreactor, tube or pipe).

### EXAMPLES

The invention will be further described in the following examples, which do not limit the scope of the invention described in the claims.

The particles used in the following examples are Cross-Linked dextran coated Iron Oxide (CLIO) particles (Table 1, entry 1).

### Example 1

### Calcium Sensor

An important class of calcium selective ionophores are based on diamide derivatives. The calcium ion recognition site in this case is the glycolic diamide backbone. A chelator was designed and synthesized based on the structure of these ionophores that can be conjugated to the amino groups on the surface of CLIO particles (Scheme 1). The conjugation to the particle was done using standard EDC/sulfo-NHS chemistry as described, for example, in Sun, E. Y.; Josephson, L.; Kelly, K. A.; Weissleder, R. Bioconjugate Chemistry, 2006, 17, 109-113.

### Synthesis

CHEL1 was synthesized using Scheme 1, which is a procedure adapted from Zhang et al., Chemical Journal on Internet, 2003; Vol. 5; and Suzuki et al., Analytical Chemistry 1995, 67, 324-334. In general, a ion-chelating molecule including a carboxylic acid reactive moiety is first synthesized. The carboxylic acid reactive moiety is then reacted with an amine functionality on a magnetic particle via carbodiimide-mediated coupling chemistry to generate an amide-linked ion-binding magnetic particle.

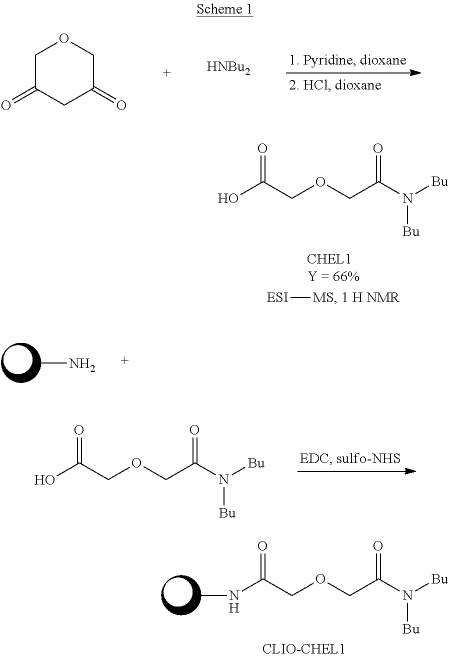

CHEL1

Diglycolic anhydride (1.0 g, 8.6 mmol) was dissolved in 8 mL 1,4-dioxane. Dibutylamine (1.45 mL, 8.6 mmol) premixed with 0.7 mL pyridine was added dropwise at 0° C. After 3 hour reaction at room temperature, solvent was evaporated and the residue was dissolved in a 1:1 dioxane/HCl solution. After evaporation of solvent and recrystallization in methanol/water (1:1), 1.39 g of pure compound CHEL1 was obtained as a white powder. Yield after recrystallization: 66%. Structure and purity confirmed by ¹H NMR and ESI-MS. NB: compound is very hygroscopic and needs to be lyophilized before each use.

CLIO-CHEL1

CHEL1 (1.1 mg, 4.5 μmol) in 50 μL DMSO was added to 1 mg of amino-CLIO in MES buffer (50 mM, 0.1 M NaCl), pH 6.0. Freshly dissolved sulfo-NHS (9.7 mg, 45 μmol) in 500 μL MES and EDC (8.6 mg, 45 μmol) in 500 μL DMSO were premixed and added to the mixture in two times with 30-minute interval. The reaction proceeded for 1 hour at room temperature and the product was purified through a Sephadex G-25 PD10-column (GE Healthcare, Uppsala, Sweden) equilibrated with PBS. The amount of chelator attached was quantified using the SPDP/TCEP method as previously reported in Sun et al., Bioconjugate Chemistry, 2006, 17, 109-113. 56 CHEL1 were found per CLIO based on 8000 Fe atom per CLIO, as described, for example, in Reynolds et al., Analytical Chemistry, 2005, 77, 814-817.

Results

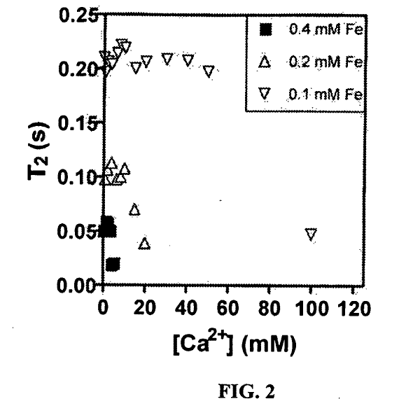

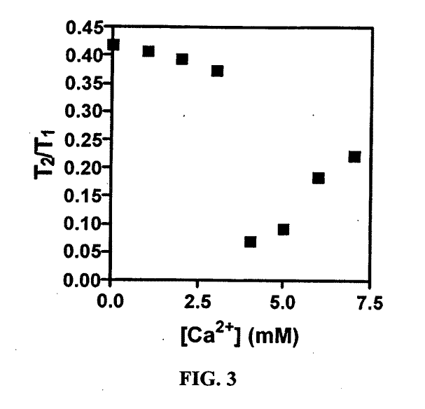

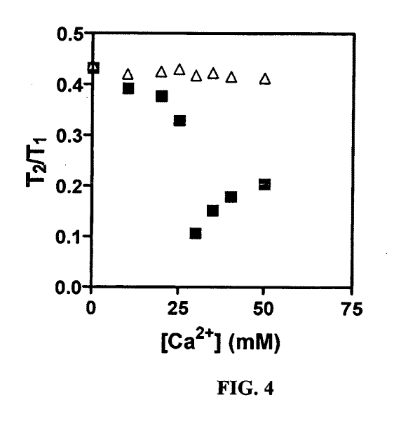

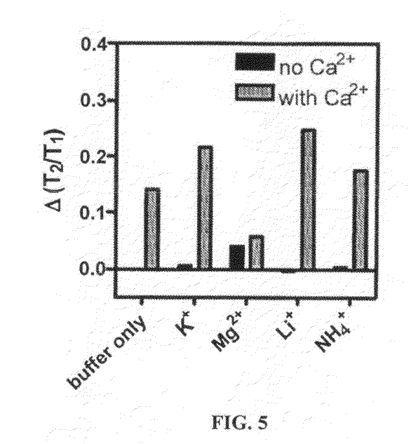

Upon addition of calcium to a suspension of CLIO-CHEL1, a change in relaxation time was observed due to clustering of the particles around the calcium ion by chelation of at least two diglycolic amide moieties. Concentration of calcium chloride in HEPES (25 mM, pH 7.2, adjusted with NaOH) was varied between 1 and 100 mM at a constant particle concentration. Three particle concentrations were used (0.1; 0.2 and 0.4 mM) in FIG. 2. The sensitivity of the test towards calcium ions increased with increasing particle concentration. By changing the particle concentration, the sensor could be tuned to the region of interest for calcium detection. For example, at a particle concentration of 0.4 mM Fe, the system's response Changed dramatically between 3 and 4 mM in calcium ion with a T₂/T₁ switch from 0.37 to 0.07 (FIG. 3). Larger loads of calcium resulted in particle precipitation. When a concentration of 0.2 mM Fe was used, the switch occurred between 20 and 30 mM in calcium ion (FIG. 4). The reversibility of the system was checked by adding EDTA (70 mM). The selectivity of the sensor was tested towards common ions (FIG. 5). All samples were in HEPES (25 mM, pH 7.2, adjusted with NaOH) and ion concentration was 28 mM.

The ability of ions to interact with ion-chelating molecules on the surfaces of particles, and cause changes in the state of clustering and T2 relaxation, was surprising and non-obvious. First, the ions must bind to ion-chelating molecules on two or more different magnetic particles rather than binding to ion-chelating molecules on the surface of the same particle. In light of the high numbers of ion-chelating molecules attached to each particle (30-70), which could result in ions binding two chelating groups on the same particle, the ion mediation of interactions between different particles is surprising. Second, unlike electrodes, particles exist free in suspension when the surface chemistry of the particles decreases (e.g., prevents) aggregation between particles in suspension. With the CLIO particles, a thick coating of polymeric dextran (10-15 nm) serves to attenuate attractions between the cores of superparamagnetic iron oxide (diameter=5-10 nm). However, if the polymer stabilizing mechanism is “too dominant,” ion binding will not affect particle aggregation. If the polymer stabilization mechanism is too weak, addition of ions will cause immediate aggregation and the particles are likely to precipitate from the suspension.

### Example 2

### Magnesium Sensor

Chelators with a malonamide backbone have been used as magnesium ion recognition in ion selective electrodes.

Synthesis CHEL2 was synthesized using Scheme 2, which is a procedure adapted from Suzuki et al., Analytical Chemistry, 1995, 67, 324-334; and Odonnell et al., Analytica Chimica Acta, 1993, 281, 129-134. In general, a ion-chelating molecule including a carboxylic acid reactive moiety is first synthesized (e.g., CHEL2_{—}1). The ion-chelating molecule is further conjugated with a valeric acid spacer (e.g., CHEL_{—}2_{—}2). The ion-chelating molecule is then reacted with an amine functionality on a magnetic particle via carbodiimide-mediated coupling chemistry to generate an amide-linked ion-binding magnetic particle.

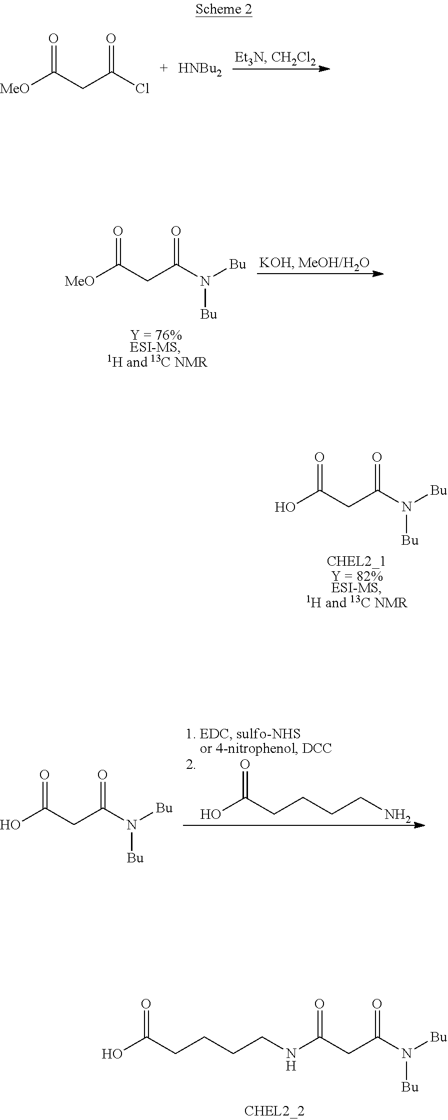

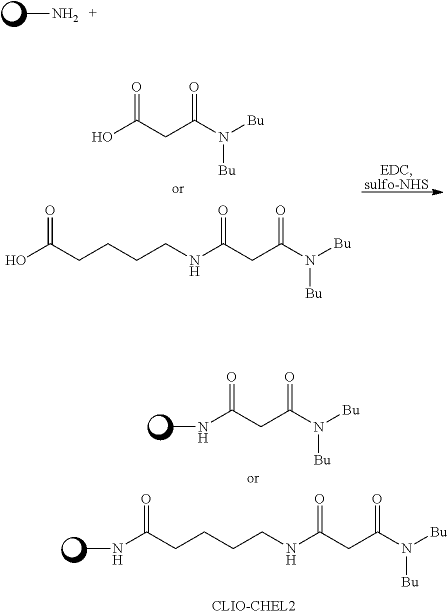

CHEL2_{—}1 Methylester

Methylchlorooxopropionate (0.8 mL, 7.3 mmol) was added dropwise at 0° C. to a mixture of dibutylamine (1.2 mL, 7.3 mmol) and triethylamine (1.0 mL, 7.3 mmol) in 8 mL dichloromethane. The reaction was left to react overnight at room temperature then was dissolved in 10 mL chloroform. After washing the mixture twice with HCl (0.1 M) then water, the organic phase was collected and dried over sodium sulfate. Product was purified by column chromatography (silica gel, ethylacetate/hexane (1:1)). 1.27 g of pure compound CHEL2_{—}1 methylester was obtained as a yellow oil. Yield: 76%. Structure and purity confirmed by ¹H NMR, ¹³C NMR and ESI-MS.

CHEL2_{—}1

Potassium hydroxide (0.33 g, 5.8 mmol) in 5 mL water was added to CHEL2_{—}1 methylester (0.74 g, 3.2 mmol) in 20 mL methanol/water (3:1). The mixture was left to react overnight at room temperature. Methanol was evaporated and the remaining water was acidified to pH 1, with HCl (1M). The oily product was extracted three times with ethyl acetate and dried over sodium sulfate. The solvent was evaporated yielding 0.57 g of product. Yield: 82%. Structure and purity confirmed by ¹H NMR, ¹³C NMR and ESI-MS. CHEL2_{—}2

The carboxylic acid group of CHEL2_{—}1 is activated by reaction with EDC/sulfo-NHS or 4-nitrophenol/DCC as described in Odonnell et al., Analytica Chimica Acta, 1993, 281, 129-134, and reacted with 5-aminovaleric acid.

CLIO-CHEL2

CHEL2_{—}1 and CHEL2_{—}2 are conjugated to CLIO using EDC/sulfo-NHS chemistry in a similar way to CHEL1 conjugation (Example 1).

### Example 3

### Copper Sensor

Thiodiglycolic acid derivatives have been used as ionophores for copper ion selective electrodes, as described, for example, in Szigeti et al., Analytica Chimica Acta 2005, 532, 129-136.

CHEL3 is synthesized according to the same procedure as for CHEL1 with thiodiglycolic anhydride as the starting material. CHEL3 is conjugated to CLIO using EDC/sulfo-NHS chemistry in a similar way to CHEL1 conjugation (Example 1).

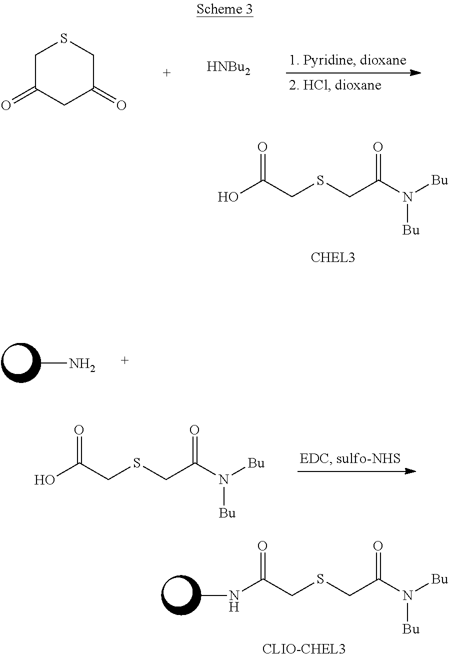

### Example 4

### Potassium Sensor

Crown ethers have been used in ion selective electrodes as well as particle-based sensors as chelators for potassium, sodium and cesium. The size of the ring determines the selectivity of the system, with two crowns forming a sandwich complex with the target ion.

CHEL4 is synthesized as described in Lin et al., Analytical Chemistry, 2005, 77, 4821-4828. CHEL4 and 6-mercaptohexanoic acid are conjugated to CLIO using N-hydroxysuccinimidyl ester of iodoacetic acid (SIA) chemistry as described, for example, in Sun et al., Bioconjugate Chemistry, 2006, 17, 109-113. In general, a ion-chelating molecule (e.g., 15-crown-5) is functionalized with a spacer (e.g., an alkylether) terminated with a reactive group, such as a thiol. The thiol-terminated ion-chelating molecule is then conjugated to an amine functionalized particle via a N-hydroxysuccinimidyl ester of iodoacetic acid to generate the ion-binding magnetic particle. 6-mercaptohexanoic acid is also conjugated to the magnetic particle to orient the ion-chelating molecule away from the magnetic particle so as to bond with an analyte molecule.

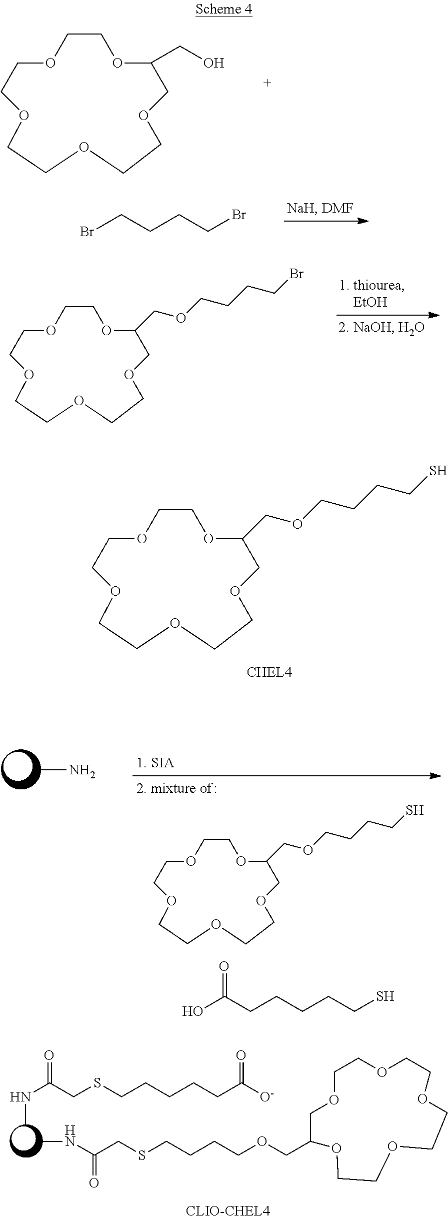

### Example 5

### Sodium Sensor

CHEL5 is synthesized as described in Lin et al., Analytical Chemistry, 2005, 77, 4821-4828. CHEL5 and 6-mercaptohexanoic acid are conjugated to CLIO using SIA chemistry as described, for example, in Sun et al., Bioconjugate Chemistry, 2006, 17, 109-113. In general, a ion-chelating molecule (e.g., 12-crown-4) is functionalized with a spacer (e.g., an alkylether) terminated with a reactive group, such as a thiol. The thiol-terminated ion-chelating molecule is then conjugated to an amine functionalized particle via a N-hydroxysuccinimidyl ester of iodoacetic acid to generate the ion-binding magnetic particle.

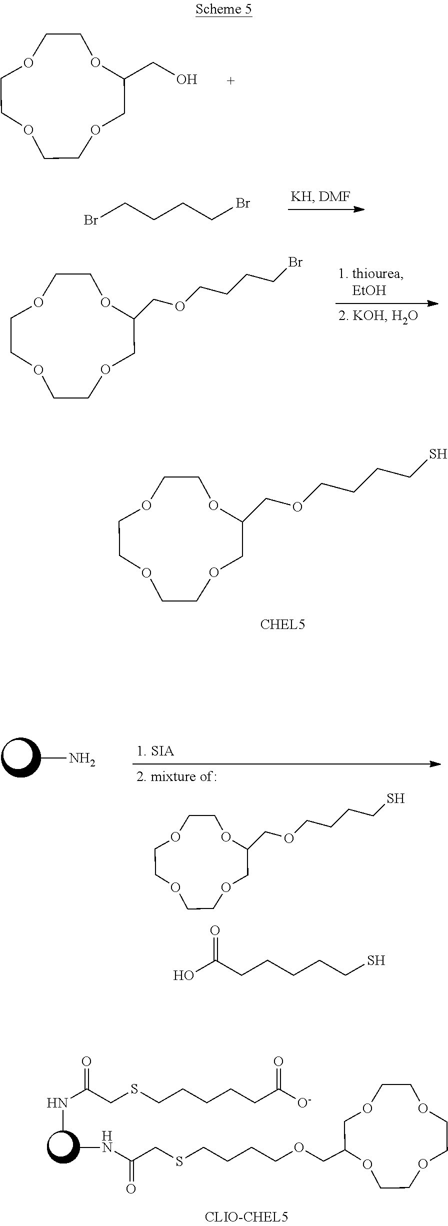

### Example 6

### Cesium Sensor

CHEL6 is synthesized using a procedure adapted from Lin et al., Analytical Chemistry, 2005, 77, 4821-4828. CHEL6 and 6-mercaptohexanoic acid are conjugated to CLIO using SIA chemistry as described, for example, in Sun et al., Bioconjugate Chemistry, 2006, 17, 109-113. In general, a ion-chelating molecule (e.g., 18-crown-6) is functionalized with a spacer (e.g., an alkylether) terminated with a reactive group, such as a thiol. The thiol-terminated ion-chelating molecule is then conjugated to an amine functionalized particle via a N-hydroxysuccinimidyl ester of iodoacetic acid to generate the ion-binding magnetic particle.

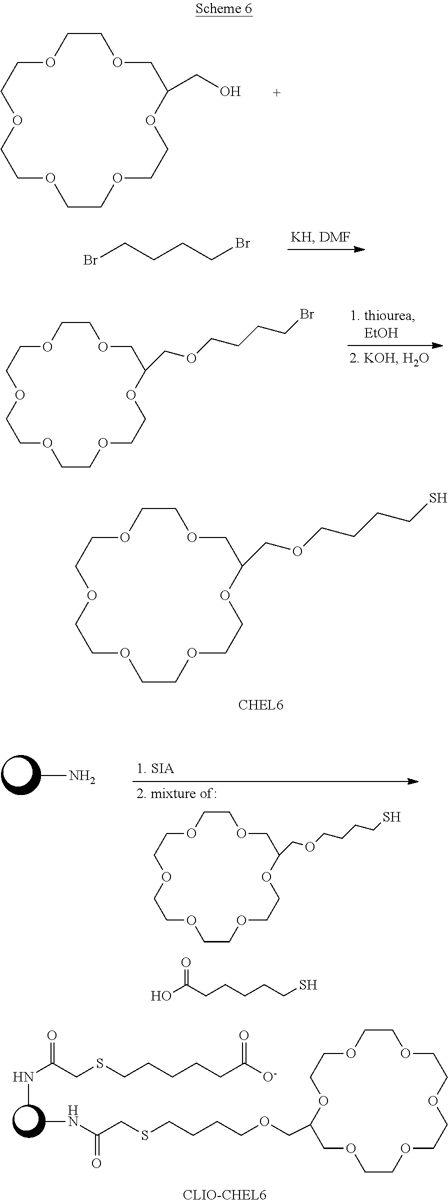

### Example 7

### Zinc Sensor

Bis(2-pyridylmethyl)ethylenediamine derivatives are known to bind selectively to zinc ion and were used as recognition moieties in gadolinium-based zinc sensors.

CHEL7 is prepared according to the procedure described in Hanaoka et al., Journal of the Chemical Society—Perkin Transactions 2 2001, 1840-1843. Conjugation to CLIO is done by first converting amino groups on CLIO to caboxylates then using EDC/sulfo-NHS chemistry as described, for example, in Sun et al., Bioconjugate Chemistry, 2006, 17, 109-113. The carboxylic acid terminated particle is then conjugated to an amine terminated ion-chelating molecule via a carbodiimide-mediated coupling reaction to generate the ion-binding magnetic particle.

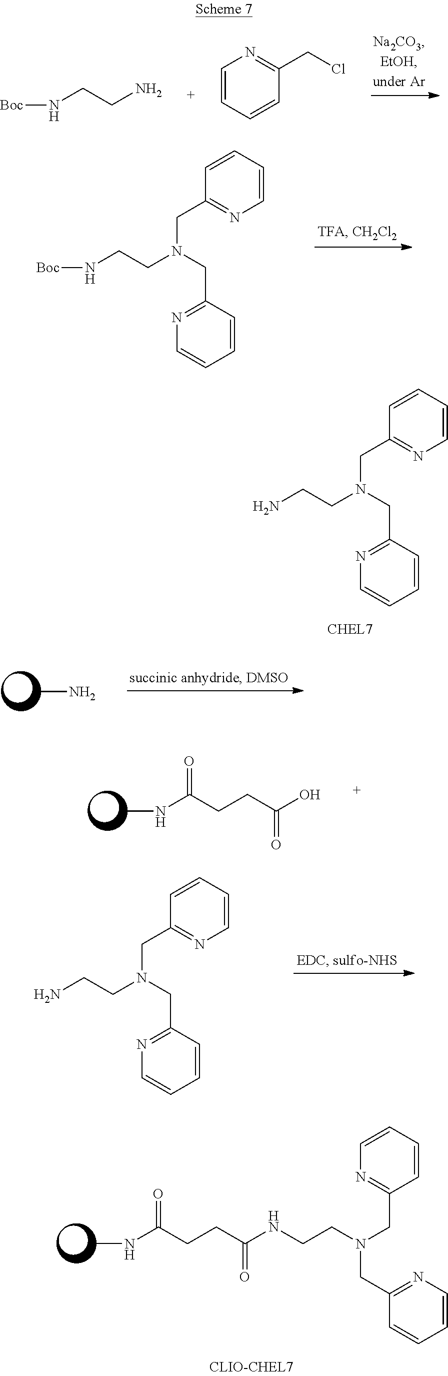

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An ion-binding particle comprising:
a magnetic particle M; and
an ion-chelating molecule Y covalently linked to the magnetic particle.

2. The ion-binding particle of claim 1, wherein the ion-binding particle comprises a moiety of Formula I covalently linked to the magnetic particle M: 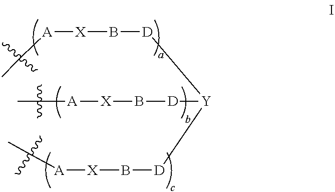
wherein
A is NHCO, CONH, S, O, or NR^{a};
X is absent, C₁₋₁₀ alkyl, C₁₋₆ haloalkyl, C₂₋₆ alkenyl, C₂₋₆ alkynyl, aryl, heteroaryl, cycloalkyl, or heterocycloalkyl, wherein said C₁₋₁₀ alkyl, C₁₋₆ haloalkyl, C₂₋₆ alkenyl, C₂₋₆ alkynyl, aryl, heteroaryl, cycloalkyl, or heterocycloalkyl is optionally substituted with 1, 2, or 3 substituents independently selected from OH, CN, amino, halo, C₁₋₆ alkyl, C₁₋₆ alkoxy, C₁₋₆ haloalkyl, and C₁₋₆ haloalkoxy;
B is absent or a spacer;
D is absent, NHCO, CONH, S, O, or NR^{a};
Y is an ion-chelating molecule;
R^{a} is H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein said C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆ alkoxy, C₁₋₆ -haloalkyl, and C₁₋₆ haloalkoxy;
a, b, and c are each independently 0 or 1; and
a+b+c is greater than or equal to 1.

3. The ion-binding particle of claim 2, wherein A is NHCO or CONH.

4. The ion-binding particle of claim 2, wherein D is absent, NHCO, or CONH.

5. The ion-binding particle of claim 2, wherein X is absent or C₁₋₁₀ alkyl.

6. The ion-binding particle of claim 2, wherein X is absent or CH₂.

7. The ion-binding particle of claim 2, wherein X is absent.

8. The ion-binding particle of claim 2, wherein the spacer is alkyl interrupted by one or more O, NR^{a}, S, SO, SO₂, C(O)O, OC(O), NHCO, CONH, SC(O), or C(O)S, said alkyl is optionally terminated with one or two O, NR^{a}, S, SO, SO₂, C(O)O, OC(O), NHCO, CONH, SC(O), or C(O)S.

9. The ion-binding particle of claim 2, wherein R^{a} is H or C₁₋₁₀ alkyl.

10. The ion-binding particle of claim 1, wherein Y is selected from:

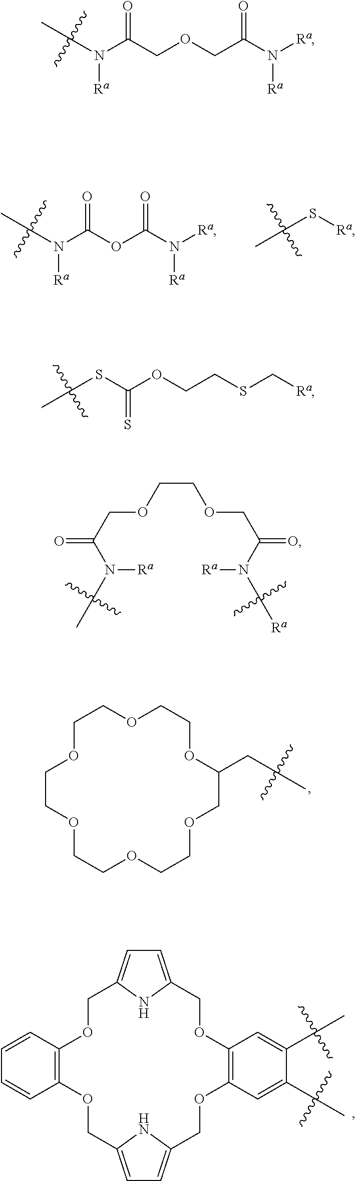

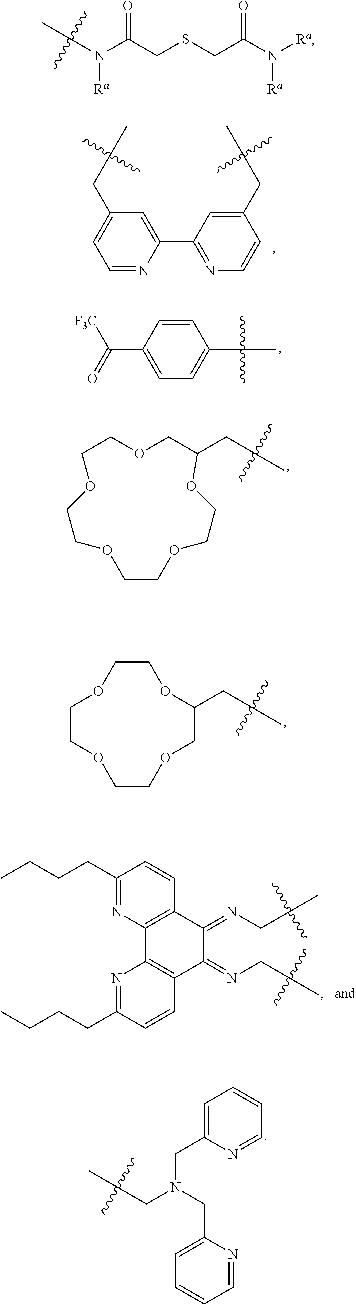

11. The ion-binding particle of claim 1, wherein Y is selected from the group consisting of a calcium-chelating molecule, a magnesium-chelating molecule, a copper-chelating molecule, a potassium-chelating molecule, a sodium-chelating molecule, a cesium-chelating molecule, a zinc-chelating molecule, and combinations thereof.

12. The ion-binding particle of claim 1, wherein the magnetic particle has a maximum dimension of less than or equal to one micron.

13. The ion-binding particle of claim 1, wherein the magnetic particle comprises a superparamagnetic material.

14. The ion-binding particle of claim 1, wherein the magnetic particle is a magnetic metal oxide.

15. The ion-binding particle of claim 1, wherein the magnetic particle has a maximum dimension of from about 15 nm to 500 nm.

16. The ion-binding particle of claim 2, wherein the particle comprises from 1 to about 200 moieties of Formula I.

17. The ion-binding particle of claim 2, wherein the particle comprises from 1 to about 100 moieties of Formula I.

18. The ion-binding particle of claim 2, wherein the particle comprises from 1 to about 75 moieties of Formula I.

19. A method of detecting a specific ion in a first sample, the method comprising
obtaining a first sample potentially comprising a specific ion;
contacting the first sample with a plurality of ion-binding particles of claim 1 for a time and under conditions sufficient to allow the formation of ion/ion-binding particle complexes;
measuring a relaxation time of the first sample; and
comparing the relaxation time of the first sample with a relaxation time of a reference;
wherein a difference between the relaxation time of the first sample and the relaxation time of the reference indicates the presence of the specific ion in the sample.

20. The method of claim 19, wherein the reference is a second sample free of the specific ion.

21. The method of claim 19, wherein the reference is contacted with a plurality of non-ion-binding particles.

22. The method of claim 19, wherein the relaxation time of the sample is converted into data, and the data of the relaxation time of the sample is compared to data corresponding to the relaxation time of the reference.

23. The method of claim 22, wherein the data of the relaxation time of the reference is a calibration curve or data corresponding to a calibration curve.

24. The method of claim 19, further comprising measuring a concentration of the detected ion, wherein the difference between the relaxation time of the first sample and the relaxation time of the reference correlates with a concentration of the ion in the first sample.

25. The method of claim 19, wherein the sample comprises an ion-binding particle concentration of at least 0.1 mM.

26. The method of claim 19, wherein the sample comprises an ion-binding particle concentration of at least 0.4 mM.

27. The method of claim 19, wherein a ratio of the relaxation time of the reference to the relaxation time of the sample decreases upon formation of ion/ion-binding particle complexes.

28. The method of claim 19, wherein formation of ion/ion-binding particle complexes is reversible upon addition of a competing chelating agent.

29. The method of claim 28, wherein the competing chelating agent is selected from the group consisting of EDTA, EGTA, DTPA, NTA acid, o-phenanthroline, dimercaptopropanol, and salicylic acid.

30. The method of claim 19, wherein formation of ion/ion-binding particle complexes is non-reversible.

31. The method of claim 19, wherein the ion/ion-binding particle complex comprises two or more ion-binding particles.

32. The method of claim 19, wherein the sample comprises a bodily fluid.

33. The method of claim 32, wherein the bodily fluid is selected from the group consisting of blood, serum, urine, and combinations thereof.

34. The method of claim 19, further comprising
obtaining a device comprising a semipermeable wall that allows passage of the specific ion but not the passage of the ion-binding particles;
enclosing the ion-binding particles within the device; and
allowing formation of the ion/ion-binding particle complexes within the device.

35. The method of claim 34, further comprising implanting the device in the subject.

36. A device comprising a plurality of ion-binding particles of claim 1 enclosed within a semipermeable wall that allows passage of an ion chelated by the ion-chelating molecule Y, but not the passage of the ion-binding particles.
